(19) United States
(12) Patent Application Publication
(10) Pub. No.: US 2025/0005387 A1
(43) Pub. Date: Jan. 2, 2025

(54) APPARATUS, METHOD AND COMPUTER PROGRAM FOR SCREENING CATHODE ACTIVE MATERIAL CANDIDATES FOR SECONDARY BATTERIES

(71) Applicant: Foundation of Soongsil University-Industry Cooperation, Seoul (KR)

(72) Inventors: Kyoungmin Min, Suwon-Si (KR); Minseon Kim, Ansan-Si (KR)

(73) Assignee: Foundation of Soongsil University-Industry Cooperation, Seoul (KR)

(21) Appl. No.: 18/404,273

(22) Filed: Jan. 4, 2024

(30) Foreign Application Priority Data
Jun. 28, 2023 (KR) 10-2023-0083652

(51) Int. Cl. G06N 5/022

(52) U.S. Cl. CPC G06N 5/022; H01M 2004/028; H01M 4/525

(57) ABSTRACT

The present disclosure relates to an apparatus for screening cathode active material candidates for secondary batteries, and may include a database constructing unit configured to receive a data-set labeled with properties of a cathode active material structure for secondary batteries; a pre-processing unit configured to pre-process a part of the data-set to a learning data-set; a prediction model generating unit configured to generate a cathode active material prediction model for predicting performance indicators of target materials that may be arranged to fit a predetermined structure based on the learning data-set; and a candidates generating unit configured to generate cathode active material candidates for secondary batteries based on a result of the cathode active material prediction model.

### CROSS-REFERENCE TO RELATED APPLICATION

This application claims priority to and the benefit of Korean Patent Application No. 10-2023-0083652, filed on Jun. 28, 2023, the disclosures of which is incorporated herein by reference in its entirety.

### FIELD

The present disclosure relates to an apparatus, a method, and a computer program for screening candidate cathode active material candidates for secondary batteries.

### BACKGROUND

Recently, the demand for secondary batteries is exploding with the rapid growth in the fields of electric vehicles and large-scale energy storage systems. Li-ion batteries (LIBs) are key materials in secondary batteries and have advantages of high energy density and capacity and long cycle life. The cathode material of the positive electrode material, which accounts for 40% of the cost of such Li-ion batteries (LIBs), plays a very important role in determining the capacity, output, and lifespan of the battery. Conventional nickel (Ni)-rich layered cathode, such as LiNiₓCo_{y}Mn_{z}O₂ (NCM) and LiNiₓCo_{y}Al_{z}O₂ (NCA), account for more than 80% α of the cathode material market, and are extensively investigated to minimize the proportion of cobalt (Co) that causes price volatility and supply chain problems.

Cobalt (Co), the main element of the cathode material, has a small amount of storage in the earth, and air pollutants may be generated during mining and smelting, which can cause environmental problems. The importance of developing a novel cathode material that satisfies the minimization of cobalt (Co) content for low manufacturing cost and the maximization of nickel (Ni) capacity for high capacity is emerging.

The development of such a novel cathode material requires several experiments and costs accordingly. Therefore, there is a need for a technology that can predict the effect of a potential material as the novel cathode material by screening it in advance.

Korean Patent Application Laid-Open No. 10-2023-0070735 (May 23, 2023) relates to a device for measuring the useful life of a reusable battery using artificial intelligence.
(Patent Document 1) Korean Patent Application Laid-Open No. 10-2023-0070735 (May 23, 2023).

### SUMMARY

An embodiment of the present disclosure relates to an apparatus, a method, and a computer program for screening cathode active material candidates capable of saving costs and time for developing cathode active materials for secondary batteries by selecting cathode active material candidates for secondary batteries including a desired performance using a cathode active material prediction model, and developing cathode active materials for secondary batteries with excellent performance.

An embodiment of the present disclosure relates to an apparatus, a method, and a computer program for screening cathode active material candidates capable of improving the performance of a cathode active material prediction model through selection of learning data.

An embodiment of the present disclosure relates to an apparatus, a method, and a computer program for screening cathode active material candidates capable of developing cathode active materials for secondary batteries with guaranteed stability by setting target materials to be developed.

The present disclosure provides an apparatus for screening cathode active material candidates for secondary batteries, the apparatus including: a database constructing unit configured to receive a data-set labeled with properties of a cathode active material structure for secondary batteries; a pre-processing unit configured to pre-process a part of the data-set to a learning data-set; a prediction model generating unit configured to generate a cathode active material prediction model for predicting performance indicators of target materials that may be arranged to fit a predetermined structure based on the learning data-set; and a candidates generating unit configured to generate cathode active material candidates for secondary batteries based on a result of the cathode active material prediction model.

According to an exemplary embodiment of the present disclosure, the pre-processing unit may perform a performance evaluation on at least one cathode active material prediction model through a verification data-set excluding the learning data-set from the data-set.

According to an exemplary embodiment of the present disclosure, the pre-processing unit may determine a ratio between the learning data-set and the verification data-set according to a result of the performance evaluation.

According to an exemplary embodiment of the present disclosure, the pre-processing unit may determine data to be excluded from the data set according to a result of the performance evaluation.

According to an exemplary embodiment of the present disclosure, the predetermined structure may be a layered structure of the cathode active material including fixed particles.

According to an exemplary embodiment of the present disclosure, the prediction model generating unit may determine a ratio between substitute particles disposed between the fixed particles and select target materials.

According to an exemplary embodiment of the present disclosure, the prediction model generating unit may select materials of represented by Chemical Formula 1 below to target materials: LiNi_{0.85}MₓN_{y}O₂ (1) (where, x+y=0.15, M and N are any one of Al, Mg, W, Sb, Ta, Y, B, Ga, Si, Ti, V, Nb, Zr, Zn, Co, Mn, La, Tb, As, Cl, Tm, Ge, Ho, Fe, Cr, Sn, Sc, Cu, Re, Mo, Se, Te, and Tl, and M and N are not overlapped).

According to an exemplary embodiment of the present disclosure, the prediction model generation unit may regenerate the cathode active material prediction model according to the ratio between the learning data-set and the verification data-set.

According to an exemplary embodiment of the present disclosure, the prediction model generating unit may generate a plurality of cathode active material prediction models corresponding to each of performance indicators to perform prediction on each of the plurality of performance indicators.

According to an exemplary embodiment of the present disclosure, the candidates generating unit may generate the cathode active material candidates for secondary batteries by excluding candidate material corresponding to a result value of the cathode active material prediction model that does not satisfy a predetermined criterion.

The present disclosure provides a method for screening cathode active material candidates for secondary batteries, the method including: receiving a data-set labeled with properties of a cathode active material structure for secondary batteries in relation to the cathode active material structure for secondary batteries; pre-processing a part of the data-set to a learning data-set; generating a cathode active material prediction model for predicting performance indicators of target materials that may be arranged to fit a predetermined structure based on the learning data-set; and generating cathode active material candidates for secondary batteries based on a result value of the cathode active material prediction model.

The computer program stored in a computer-readable recording medium that may execute a method for screening cathode active material candidates for secondary batteries according to an embodiment of the present disclosure may include receiving a data-set labeled with properties of a cathode active material structure for secondary batteries in relation to the cathode active material structure for secondary batteries; pre-processing a part of the data-set to a learning data-set; generating a cathode active material prediction model for predicting performance indicators of target materials that may be arranged to fit a predetermined structure based on the learning data-set; and generating cathode active material candidates for secondary batteries based on a result value of the cathode active material prediction model.

The disclosed technology may have the following effects. However, since the specific embodiment does not mean that the following effects should be included in any case or only the following effects, the scope of the disclosed technology should not be construed as being limited thereto.

According to an embodiment of the present disclosure, the apparatus, the method, and the computer program for screening cathode active material candidates for secondary batteries can save costs and time for developing cathode active materials for secondary batteries by selecting cathode active material candidates for secondary batteries including a desired performance using a cathode active material prediction model, and develop cathode active materials for secondary batteries with excellent performance.

According to an embodiment of the present disclosure, the apparatus, the method, and the computer program for screening cathode active material candidates for secondary batteries can improve the performance of a cathode active material prediction model through selection of learning data.

According to an embodiment of the present disclosure, the apparatus, the method, and the computer program for screening cathode active material candidates for secondary batteries can develop cathode active materials for secondary batteries with guaranteed stability by setting target materials to be developed.

### BRIEF DESCRIPTION OF THE DRAWINGS

FIG. 1 is a diagram illustrating a system for screening cathode active material candidates for secondary batteries according to an embodiment of the present disclosure.

FIG. 2 is a diagram illustrating an apparatus for screening cathode active material candidates for secondary batteries according to an embodiment of the present disclosure.

FIG. 3 is a diagram illustrating describing a functional configuration of an apparatus for screening cathode active material candidates for secondary batteries according to an embodiment of the present disclosure.

FIG. 4 is a flowchart illustrating a sequence of a method for screening cathode active material candidates for secondary batteries according to an embodiment of the present disclosure.

FIGS. 5A, 5B, and 5C show diagrams illustrating a result of performance evaluation for a cathode active material prediction model according to an embodiment of the present disclosure.

FIG. 6 is a diagram illustrating performance of an arbitrary cathode active material prediction model depending on a ratio of a learning data-set according to an embodiment of the present disclosure.

FIGS. 7A and 7B show diagrams illustrating a result of performance evaluation for a cathode active material prediction model according to a data-set pre-processing according to an embodiment of the present disclosure.

FIG. 8 is a diagram illustrating a step of selecting cathode active material candidates according to an embodiment of the present disclosure.

### DETAILED DESCRIPTION

The present disclosure is only a structural and functional embodiment, and thus the scope of the present disclosure should not be construed as being limited by the embodiments described in the present disclosure. That is, the embodiments may be variously modified and have various forms, and thus the scope of the present disclosure should be understood to include equivalents that may realize the technical idea. Further, the objects or effects presented in the present disclosure do not mean that the specific embodiments should include the present disclosure or only the present disclosure, and thus the scope of the present disclosure should not be construed as being limited thereto.

Meanwhile, the meaning of the terminology described in the present application should be understood as follows.

The terms “first”, “second”, and the like are used to distinguish one element from another element, and the scope of the present disclosure should not be limited by these terms. For example, a first element may be referred to as a second element, and similarly, a second element may be referred to as a first element.

When an element is referred to as being “connected to” another element, it should be understood that the element may be directly connected to the other element, but the other element may be present in the middle. On the other hand, when an element is referred to as being “directly connected to” another element, it should be understood that the other element does not exist in the middle. Meanwhile, other expressions that describe a relationship between elements, that is, “between” and “directly between” or “adjacent to” and “directly adjacent to” should be interpreted as well.

It should be understood that a singular expression should be interpreted as including a plural expression unless the context clearly indicates otherwise, and that terms “include” or “have” specify the presence of stated features, numbers, steps, operations, elements, parts or combinations thereof, and do not preclude the presence or addition of one or more other features, numbers, steps, operations, elements, parts or combinations thereof.

In each step, an identifier (e.g., A, B, C, and the like) is used for convenience of description, and the identifier does not describe the order of each step, and each step may occur differently from the specified order unless the context clearly indicates a specific order. That is, each step may occur in the same order as the specified order, may be performed substantially simultaneously, or may be performed in the reverse order.

The present disclosure may be embodied as computer-readable code on a computer-readable recording medium, and the computer-readable recording medium includes all kinds of recording devices in which data readable by a computer system is stored. Examples of the computer-readable recording medium include ROM, RAM, CD-ROM, magnetic tape, floppy disk, optical data storage device, and the like, and also include a carrier wave (for example, transmission through the Internet). In addition, the computer-readable recording medium is distributed over a networked computer system, so that computer-readable codes can be stored and executed in a distributed manner.

Unless otherwise defined, all terms used herein have the same meaning as commonly understood by one of ordinary skill in the art to which the present disclosure pertains. It should be understood that terms defined in a generally used dictionary are interpreted as having the same meaning as those in the context of the related art, and may not be interpreted as having an idealized or overly formal meaning unless expressly so defined herein.

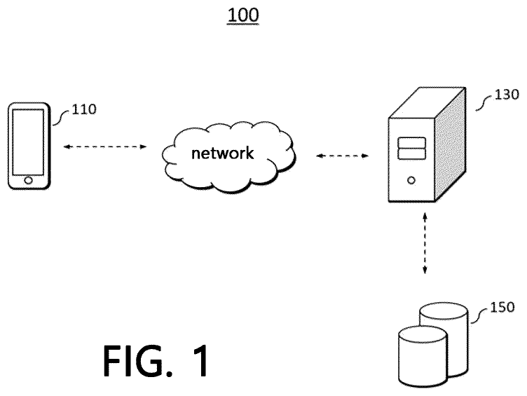

FIG. 1 is a diagram illustrating a system 100 for screening cathode active material candidates for secondary batteries according to an embodiment of the present disclosure.

Referring to FIG. 1, the system 100 for screening cathode active material candidates for secondary batteries may include a user terminal 110, an apparatus 130 for screening cathode active material candidates for secondary batteries, and a database 150.

The user terminal 110 may be implemented as a smartphone or a wearable device capable of identifying data generated and metadata analyzed according to an operation of receiving a data-set labeled with properties of a cathode active material structure for the secondary batteries through the apparatus 130 screening cathode active material candidates, pre-processing the corresponding data-set as a learning data-set, generating a cathode active material prediction model based on the pre-processing, and selecting the cathode active material candidates for the secondary batteries, but is not limited thereto, and may be implemented as various devices such as a tablet PC. The user terminal 110 may be connected to the apparatus 130 screening cathode active material candidates for the secondary batteries through a network, and the plurality of user terminals 110 may be connected to the apparatus 130 screening cathode active material candidates for the secondary batteries at the same time.

The apparatus 130 for screening cathode active material candidates for secondary batteries may be implemented as a server corresponding to a computer or program that sequentially performs operations for receiving a data-set labeled with properties of a cathode active material structure for secondary batteries, pre-processing the corresponding data-set as a learning data-set, generating a cathode active material prediction model based on the pre-processing, and selecting the cathode active material candidates for secondary batteries. The apparatus 130 for screening cathode active material candidates for secondary batteries may be wirelessly connected to the user terminal 110 through Bluetooth, Wi-Fi, a communication network, or the like, and may exchange data with the user terminal 110 through the network.

The apparatus 130 for screening cathode active material candidates for secondary batteries may be provided to be included in a computer-readable recording medium by tangibly embodying a program of instructions for implementing the same. In other words, the apparatus 130 for screening cathode active material candidates for secondary batteries may be implemented in the form of program instructions that may be executed by various computer means and may be recorded on the computer-readable recording medium. In addition, the apparatus 130 for screening cathode active material candidates for secondary batteries may include a computer program that sequentially performs operations for receiving a data-set labeled with properties of a cathode active material structure for secondary batteries, pre-processing the corresponding data-set as a learning data-set, generating a cathode active material prediction model based on the pre-processing, and selecting the cathode active material candidates for secondary batteries, and the computer program may be stored in the computer-readable recording medium.

The database 150 may correspond to a storage device for storing various information generated through a process of receiving a data-set labeled with properties of a cathode active material structure for secondary batteries, pre-processing the corresponding data-set as a learning data-set, generating a cathode active material prediction model based on the pre-processing, and selecting the cathode active material candidates for secondary batteries.

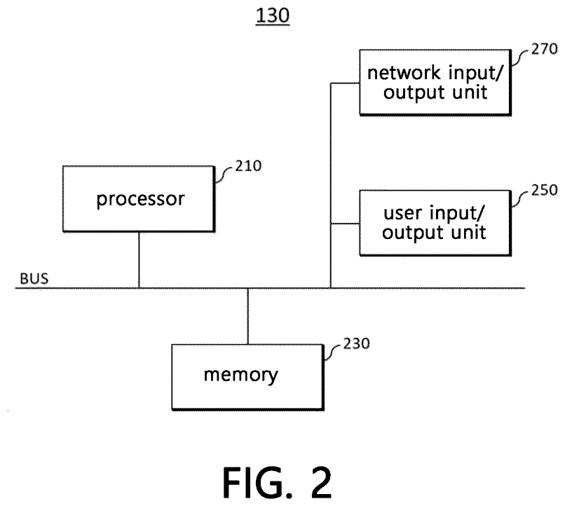

FIG. 2 is a diagram illustrating an apparatus 130 for screening cathode active material candidates for secondary batteries according to an embodiment of the present disclosure.

Referring to FIG. 2, the apparatus 130 for screening cathode active material candidates for secondary batteries may be implemented by including a processor 210, a memory 230, a user input/output unit 250, and a network input/output unit 270.

The processor 210 may execute a procedure for receiving a data-set labeled with properties of a cathode active material structure for secondary batteries, pre-processing the corresponding data-set as a learning data-set, generating a cathode active material prediction model based on the pre-processing, and selecting the cathode active material candidates for secondary batteries, manage the memory 230 read or written throughout the process, and a synchronization time between volatile memory and non-volatile memory in the memory 230 may be scheduled. The processor 210 may control the overall operation of the apparatus 130 for screening cathode active material candidates for secondary batteries, and may be electrically connected to the memory 230, the user input/output unit 250, and the network input/output unit 270 to control data flow therebetween. The processor 210 may be implemented as a central processing unit (CPU) of the apparatus 130 for screening cathode active material candidates for secondary batteries.

The memory 230 may include an auxiliary memory device implemented as a non-volatile memory such as a solid state drive (SSD) or a hard disk drive (HDD) and used to store the overall data required for the apparatus 130 for screening cathode active material candidates for secondary batteries, and may include a main memory device implemented as a volatile memory such as a random access memory (RAM).

The user input/output unit 250 may include an environment for receiving a user input and an environment for outputting specific information to a user. For example, the user input/output unit 250 may include an input device including an adapter such as a touch pad, a touch screen, an on-screen keyboard, or a pointing device, and an output device including an adapter such as a monitor or a touch screen. In an embodiment, the user input/output unit 250 may correspond to a computing device connected via remote access, and in such a case, the apparatus 130 for screening cathode active material candidates for secondary batteries may be performed as a server.

The network input/output unit 270 may include an environment for connecting to an external device or system through the network, and may include, for example, an adapter for communication such as a local area network (LAN), a metropolitan area network (MAN), a wide area network (WAN), and a value added network (VAN).

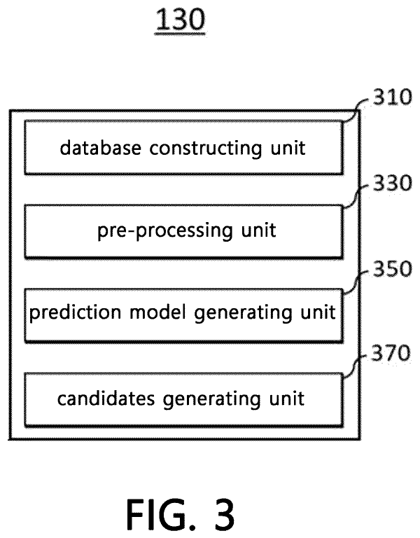

FIG. 3 is a diagram illustrating describing a functional configuration of an apparatus for screening cathode active material candidates 130 for secondary batteries according to an embodiment of the present disclosure.

Referring to FIG. 3, the apparatus 130 for screening cathode active material candidates for secondary batteries may include a database constructing unit 310, a pre-processing unit 330, a prediction model generating unit 350, and a candidates generating unit 370.

The database constructing unit 310 may receive a data-set labeled with properties of a cathode active material structure for secondary batteries. In detail, the database constructing unit 310 may receive properties including physical/chemical properties such as a charge/discharge chemical formula, a working ion, a capacity, an energy density, an average voltage, a volume change according to use, a space group number, a degree of delithiation, and a capacity per unit weight with respect to a structure capable of being used as a cathode active material for secondary batteries. Here, the labeling herein may be a feature of the properties of a particular cathode active material structure.

The pre-processing unit 330 may pre-process a part of the data-sets as a learning data-set. For example, the pre-processing unit 330 may pre-process the space group information and the degree of delithiation for a cathode active material structure for a specific secondary battery as a learning data-set, and pre-process the working ions for cathode active material structure for a specific secondary battery as those that are not the learning data-set. The pre-processing process by the pre-processing unit 330 may be different for each voltage-related cathode active material prediction model and volume-related cathode active material prediction model, as described below.

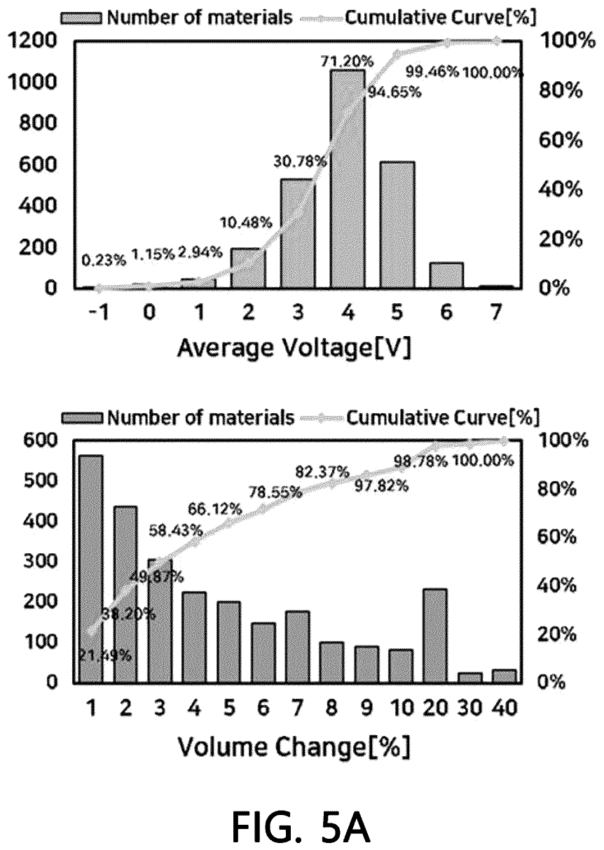

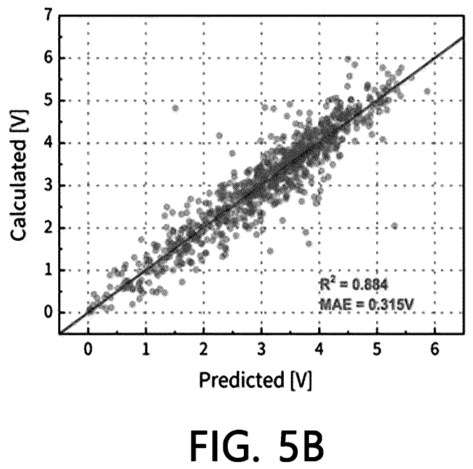

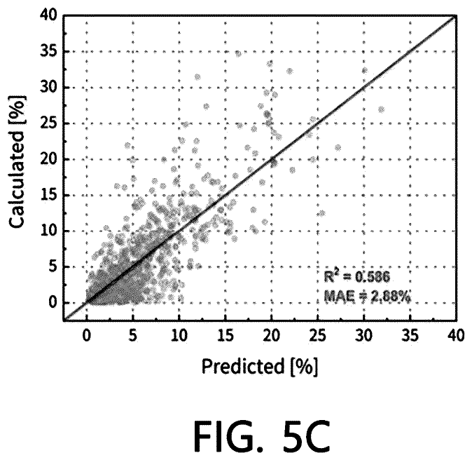

FIG. 5 is a diagram illustrating a result of performance evaluation for a cathode active material prediction model according to an embodiment of the present disclosure. Referring to FIG. 5, in an embodiment, the pre-processing unit 330 may perform performance evaluation on at least one or more cathode active material prediction model through the verification data-set excluding the learning data-set from the data-set. The pre-processing unit 330 may classify the learning data-set and the verification data-set from the data-set. That is, the pre-processing unit 330 may classify the learning data-set used for learning the cathode active material prediction model and the verification data-set used for verifying the cathode active material prediction model by dividing them into the data-set in advance. Here, the pre-processing unit 330 may classify the learning data-set and the verification data-set by dividing them into different types for each of the voltage-related cathode active material prediction model and the volume-related cathode active material prediction model, or may classify the same.

The pre-processing unit 330 may perform performance evaluation on the cathode active material prediction model through the verification data-set, which may be performed by comparing the result of each cathode active material prediction model with the labeling value of the verification data-set. Here, a statistical technique widely used may be used as a method of comparing the result value of the cathode active material prediction model with the labeling value of the verification data-set, and as an example, a coefficient of determination (R-Squared) method or a mean absolute error (MAE) method may be used. For example, the pre-processing unit 330 may calculate a distribution of result values of each of the voltage-related cathode active material prediction model and the volume-related cathode active material prediction model as shown in FIG. 5A. For another example, the pre-processing unit 330 may calculate a determination coefficient value and an MAE value for each of the voltage-related cathode active material prediction model and the volume-related cathode active material prediction model as shown in FIGS. 5B and 5C. When the pre-processing unit 330 does not satisfy a predetermined criterion according to the result of performance evaluation for each cathode active material prediction model, the pre-processing unit 330 may increase the performance of the cathode active material prediction model by adjusting the ratio between the learning data-set and the verification data-set and providing the learning data-set and the verification data-set except for the specific data-set. This will be described in detail below.

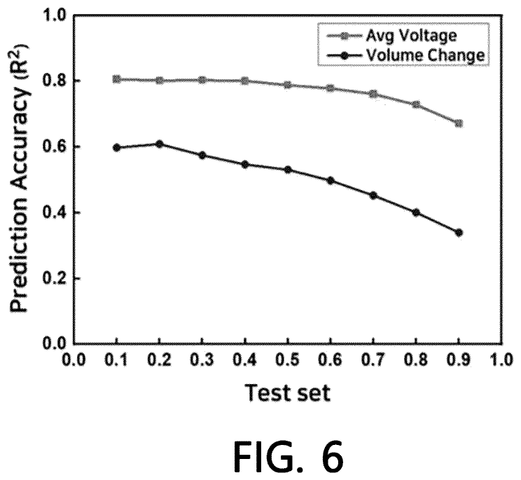

FIG. 6 is a diagram illustrating performance of an arbitrary cathode active material prediction model depending on a ratio of a learning data-set according to an embodiment of the present disclosure. Referring to FIG. 6, in an embodiment, the pre-processing unit 330 may determine a ratio between the learning data-set and the verification data-set according to a result of the performance evaluation. The pre-processing unit 330 may evaluate the performance of the cathode active material prediction model according to the ratio of the verification data-set, and determine the ratio between the learning data-set and the verification data-set based on the result value of the performance evaluation. As described above, the pre-processing unit 330 may determine the ratio between the learning data-set and the verification data-set for each cathode active material prediction model in the same or different way, and when the ratio is determined in the same, the pre-processing unit 330 may determine the ratio between the learning data-set and the verification data-set as the ratio of the verification data-set indicating the highest result value by summing the performance evaluation values of each cathode active material prediction model. For example, the pre-processing unit 330 may evaluate the performance of the cathode active material prediction model by creating a plurality of cathode active material prediction models according to a specific ratio between the learning data-set and the verification data-set. That is, the pre-processing unit 330 may test the performance of 10 voltage-related cathode active material prediction models by generating 10 voltage-related cathode active material prediction models having a ratio of the verification data-set of 10%, and may test the performance of 10 voltage-related cathode active material prediction models by generating 10 voltage-related cathode active material prediction models having a ratio of the verification data-set of 20%. In this case, the cathode active material prediction model used for testing is not the final cathode active material prediction model of the present disclosure, and as described below, the prediction model generating unit 350 may generate the final cathode active material prediction model by applying a ratio between the learning data-set and the verification data-set finally determined based on the data-set after excluding data to be excluded from the data-set.

In an embodiment, the pre-processing unit 330 may use 80% of the learning data-set and 20% of the verification data-set for the voltage-related cathode active material prediction model, and 70% of the learning data-set and 30% of the verification data-set for the volume-related cathode active material prediction model, which may vary according to performance of the prediction model, but is not limited to a specific number.

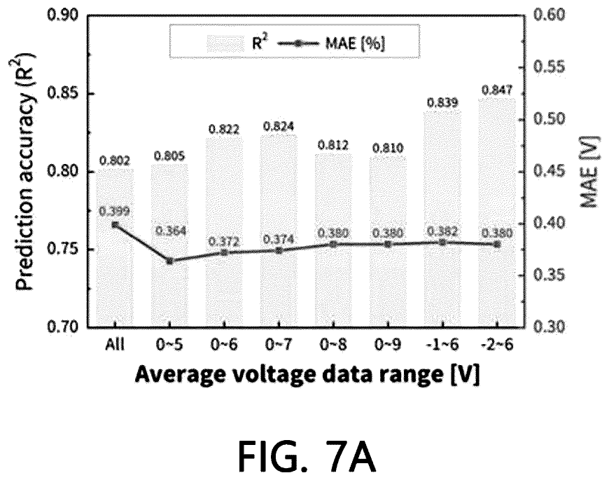

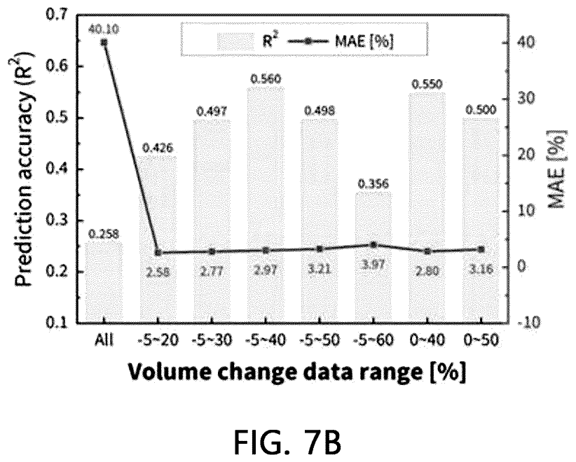

FIG. 7 is a diagram illustrating a result of performance evaluation for a cathode active material prediction model according to a data-set pre-processing according to an embodiment of the present disclosure. Referring to FIG. 7, in an embodiment, the pre-processing unit 330 may determine data to be excluded from the data-set according to a result of performance evaluation. Referring to FIG. 7A, it can be seen that the voltage-related cathode active material prediction model has excellent prediction performance when a data-set having an average voltage of −2V to 6V is used due to a property of each cathode active material structure. Referring to FIG. 7B, it can be seen that the volume-related cathode active material prediction model has excellent prediction performance when a data-set having a volume change of −5% to 40% is used due to a property of each cathode active material structure. The pre-processing unit 330 may process the data-set by limiting the labeled value of a specific property to a specific range in the measured sampled data-set. For example, the pre-processing unit 330 may process the data-set by limiting a value related to a property to be predicted by the cathode active material prediction model to a specific range. In an embodiment, in order to generate a voltage-related cathode active material prediction model among 4,394 total data-sets, the pre-processing unit 330 may determine 4,375 data-sets having an average voltage of −2V to 6V as data-sets, and may divide the learning data-sets and the verification data-sets by a predetermined ratio among the 4,375 data-sets. In another embodiment, in order to generate a volume-related cathode active material prediction model among 4,090 total data-sets, the pre-processing unit 330 may determine 4,090 data-sets having −5% to 40% as data-sets, and may divide the learning data-sets and the verification data-sets by a predetermined ratio among 4,090 data-sets.

The prediction model generating unit 350 may generate a cathode active material prediction model that predicts performance indicators of target materials that may be arranged to fit a predetermined structure based on the learning data-set. The prediction model generating unit 350 may generate the cathode active material prediction model as a model for predicting one performance indicator of target materials. In other words, one cathode active material prediction model may be a model for predicting one performance indicator of target materials, and the prediction model generating unit 350 may generate a plurality of cathode active material prediction models to predict various performance indicators of target materials, and generate a model group for predicting various performance indicators of a specific target material by ensemble the plurality of cathode active material prediction models.

In this case, the predetermined structure may be a layered structure of the cathode active material including fixed particles. That is, when the cathode active material for secondary batteries to be predicted is set, the prediction model generating unit 350 may determine a predetermined structure by fixing a specific particle and preliminarily determining a position of a particle that may be changed. For example, the prediction model generating unit 350 may determine the predetermined structure by fixing Li and O₂ at both ends of the chemical formula and changing only intermediate particles.

In an embodiment, the prediction model generating unit 350 may select target materials by determining a ratio between substitute particles disposed between fixed particles. For example, the prediction model generating unit 350 may place Li, Ni, and O2 as fixed particles at both ends of the chemical formula, determine substitute particles as Al and Mg, and select LiNi_{0.85}Al_{0.1}Mg_{0.05}O₂ as a target material. In addition, the prediction model generating unit 350 may select target materials by determining one substitute particle disposed between fixed particles as one.

In an embodiment, the prediction model generating unit 350 may determine materials of Chemical Formula 1 as target materials.

NiNi_{0.85}MₓN_{y}O₂ (1)
(where, x+y=0.15, M and N are any one of Al, Mg, W, Sb, Ta, Y, B, Ga, Si, Ti, V, Nb, Zr, Zn, Co, Mn, La, Tb, As, Cl, Tm, Ge, Ho, Fe, Cr, Sn, Sc, Cu, Re, Mo, Se, Te, and Tl, and M and N are not overlapped).

Here, the prediction model generating unit 350 may discretely determine x and y values in advance. For example, the prediction model generating unit 350 may adjust the number of target materials by predetermining x:y to be 0.15:0, 0.1:0.05, 0.075:0.075, and 0.05:0.1.

In an embodiment, the prediction model generating unit 350 may regenerate the cathode active material prediction model according to a ratio between the learning data-set and the verification data-set. As described above, the prediction model generating unit 350 may regenerate the cathode active material prediction model according to the optimized ratio between the learning data-set and the verification data-set. In addition, the prediction model generation unit 350 may regenerate the cathode active material prediction model based on the learning data set divided according to the optimized ratio of the learning data set and the verification data set in the pre-processed data set after excluding the data to be excluded from the data set by the pre-processing unit 330 to determine the cathode active material prediction model as the final prediction model. This process may be applied to each cathode active material prediction model as described above.

In an embodiment, the prediction model generating unit 350 may generate a plurality of cathode active material prediction models corresponding to each of the performance indicators so as to perform prediction on each of the plurality of performance indicators. For example, the prediction model generating unit 350 may generate a voltage-related cathode active material prediction model for predicting an average voltage of the cathode active material and a volume-related cathode active material prediction model for predicting a volume change during charge and discharge of the cathode active material. That is, the prediction model generating unit 350 may generate a corresponding cathode active material prediction model for performing prediction on each of the performance indicators. As described above, the prediction model generating unit 350 may generate a plurality of cathode active material prediction models to predict various performance indicators of target materials, and generate a model group for predicting various performance indicators of a specific target material by ensemble the models.

The candidates generating unit 370 may generate cathode active material candidates for secondary batteries based on a result value of the cathode active material prediction model. For example, the candidates generating unit 370 may generate a top 20% model of the result value of the cathode active material prediction model as the cathode active material candidates for secondary batteries. In addition, when there are several result values of the plurality of cathode active material prediction models, the candidates generating unit 370 may generate a cathode active material candidates in a manner that a reference is set only for a specific result value and a reference is not set for the remaining result values. This will be described in detail below.

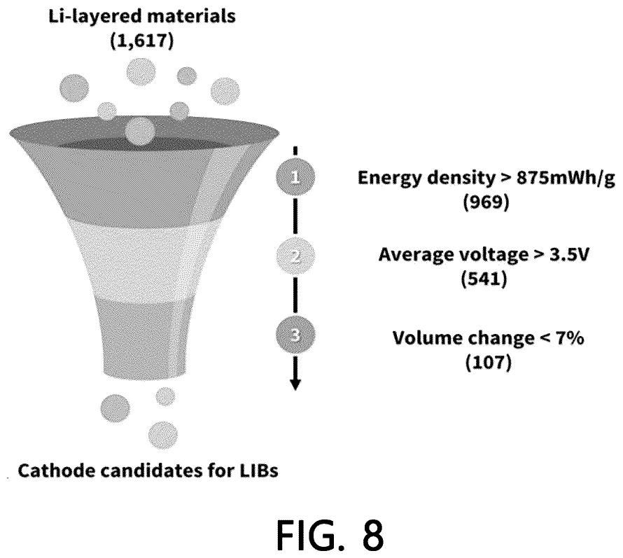

FIG. 8 is a diagram illustrating a step of selecting cathode active material candidates according to an embodiment of the present disclosure. Referring to FIG. 8, in an embodiment, the candidates generating unit 370 may generate cathode active material candidates for secondary batteries by excluding a candidate material corresponding to a result value of the cathode active material prediction model that does not satisfy a predetermined criterion. Referring to FIG. 8, the candidates generator 370 according to an embodiment may determine only a material having an energy density of 875 mWh/g, an average voltage of more than 3.5 V, and a volume change of less than 7% during charging/discharging as cathode active material candidates for 1,617 target materials. For example, the candidates generating unit 370 may determine a target material having a high or low value based on an average value of each result value among the result values of the voltage-related cathode active material prediction model and the volume-related cathode active material prediction model as cathode active material candidates. For another example, the candidates generating unit 370 may determine cathode active material candidates based on only some result values among the result values of an ensemble model of cathode active material prediction models including the voltage-related cathode active material prediction model and the volume-related cathode active material prediction model. In more detail, the candidates generating unit 370 may determine cathode active material candidates based on the average voltage value related to the voltage-related cathode active material prediction model among the result values of an ensemble model of cathode active material prediction models including the voltage-related cathode active material prediction model and the volume-related cathode active material prediction model. In addition, when the candidates generating unit 370 includes a cathode active material prediction model related to other properties other than the voltage-related cathode active material prediction model and the volume-related cathode active material prediction model, the candidates generating unit 370 may determine cathode active material candidates based on average voltage and volume change.

In an embodiment, the candidates generating unit 370 may verify a result of the cathode active material prediction model. Unlike the pre-processing unit 330, the candidates generating unit 370 may verify a result of the cathode active material prediction model by comparing the calculated value computed through Density Functional Theory (DFT) operation with the result of the cathode active material prediction model without a separate verification data-set.

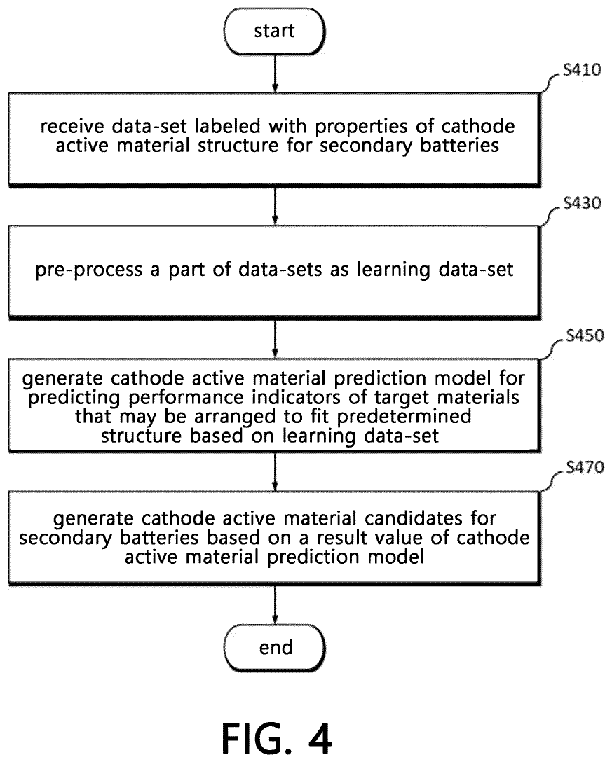

FIG. 4 is a flowchart illustrating a sequence of a method for screening cathode active material candidates for secondary batteries according to an embodiment of the present disclosure.

Referring to FIG. 4, the method for screening cathode active material candidates for secondary batteries may receive a data-set labeled with properties of a cathode active material structure for secondary batteries in relation to the cathode active material structure for secondary batteries through the database constructing unit 310 (Step 410).

The method for screening cathode active material candidates for secondary batteries may pre-process a part of the data-sets as a learning data-set through the pre-processing unit 330 (Step 430).

The method for screening cathode active material candidates for secondary batteries may generate a cathode active material prediction model for predicting performance indicators of target materials that may be arranged to fit a predetermined structure based on a learning data-set through the prediction model generating unit 350. (Step 450).

The method for screening cathode active material candidates for secondary batteries may generate cathode active material candidates for secondary batteries based on a result value of a cathode active material prediction model through the candidates generating unit 370 (Step 470).

Although the present disclosure has been described with reference to the preferred embodiments of the present disclosure, it will be understood that those skilled in the art can modify and change the present disclosure in various ways without departing from the spirit and scope of the present disclosure as set forth in the appended claims.

### EXPLANATION OF REFERENCE NUMERALS

100: system for screening cathode active material candidates for secondary batteries
110: user terminal
150: database
210: processor
230: memory
250: user input/output unit
270: network input/output unit
310: database constructing unit
330: pre-processing unit
350: prediction model generating unit
370: candidates generating unit

What is claimed is:

1. An apparatus for screening cathode active material candidates for secondary batteries, the apparatus comprising:
a database constructing unit configured to receive a data-set labeled with properties of a cathode active material structure for secondary batteries;
a pre-processing unit configured to pre-process a part of the data-set to a learning data-set;
a prediction model generating unit configured to generate a cathode active material prediction model for predicting performance indicators of target materials that may be arranged to fit a predetermined structure based on the learning data-set; and
a candidates generating unit configured to generate cathode active material candidates for secondary batteries based on a result of the cathode active material prediction model.

2. The apparatus of claim 1, wherein the pre-processing unit configured to:
perform a performance evaluation on at least one cathode active material prediction model through a verification data-set excluding the learning data-set from the data-set.

3. The apparatus of claim 2, wherein the pre-processing unit configured to:
determine a ratio between the learning data-set and the verification data-set according to a result of the performance evaluation.

4. The apparatus of claim 2, wherein the pre-processing unit configured to:
determine data to be excluded from the data set according to a result of the performance evaluation.

5. The apparatus of claim 1, wherein the predetermined structure is a layered structure of the cathode active material including fixed particles.

6. The apparatus of claim 5, wherein the prediction model generating unit configured to:
determine a ratio between substitute particles disposed between the fixed particles and select target materials.

7. The apparatus of claim 1, wherein the prediction model generating unit configured to:
select materials of represented by Chemical Formula 1 below to target materials:
LiNi_{0.85}MₓN_{y}O₂ (1)
(where, x+y=0.15, M and N are any one of Al, Mg, W, Sb, Ta, Y, B, Ga, Si, Ti, V, Nb, Zr, Zn, Co, Mn, La, Tb, As, Cl, Tm, Ge, Ho, Fe, Cr, Sn, Sc, Cu, Re, Mo, Se, Te, and Tl, and M and N are not overlapped).

8. The apparatus of claim 3, wherein the prediction model generation unit configured to:
regenerate the cathode active material prediction model according to the ratio between the learning data-set and the verification data-set.

9. The apparatus of claim 1, wherein the prediction model generating unit configured to:
generate a plurality of cathode active material prediction models corresponding to each of performance indicators to perform prediction on each of the plurality of performance indicators.

10. The apparatus of claim 1, wherein the candidates generating unit configured to:
generate the cathode active material candidates for secondary batteries by excluding candidate material corresponding to a result value of the cathode active material prediction model that does not satisfy a predetermined criterion.

11. A method for screening cathode active material candidates for secondary batteries, the method comprising:
receiving a data-set labeled with properties of a cathode active material structure for secondary batteries in relation to the cathode active material structure for secondary batteries;
pre-processing a part of the data-set to a learning data-set;
generating a cathode active material prediction model for predicting performance indicators of target materials that may be arranged to fit a predetermined structure based on the learning data-set; and
generating cathode active material candidates for secondary batteries based on a result value of the cathode active material prediction model.

12. A computer program stored in a computer-readable recording medium to be coupled to a computing device and to perform the steps of:
receiving a data-set labeled with properties of a cathode active material structure for secondary batteries in relation to the cathode active material structure for secondary batteries;
pre-processing a part of the data-set to a learning data-set;
generating a cathode active material prediction model for predicting performance indicators of target materials that may be arranged to fit a predetermined structure based on the learning data-set; and
generating cathode active material candidates for secondary batteries based on a result value of the cathode active material prediction model.
